(19) United States
(12) Patent Application Publication
(10) Pub. No.: US 2011/0098522 A1
(43) Pub. Date: Apr. 28, 2011

(54) Particle Beam Treatment System

(75) Inventors: Jurgen Drees, Wuppertal (DE); Helmut Piel, Wuppertal (DE)

(73) Assignee: CRYOELECTRA GMBH, Wuppertal (DE)

(21) Appl. No.: 12/919,583

(22) PCT Filed: Feb. 27, 2009
(86) PCT No.: PCT/EP2009/052350

§ 371 Date: Dec. 6, 2010

(30) Foreign Application Priority Data
Feb. 27, 2008 (DE) 10 2008 011 326.3

(51) Int. Cl. A61N 5/00

(52) U.S. Cl. 600/1

(57) ABSTRACT

A particle beam treatment system comprising a beam generating unit for generating and adjusting the kinetic energy of a beam of charged particles, comprising at least two beam guide units for feeding the beam of charged particles to a treatment location associated with the respective beam guide unit, wherein each beam guide unit comprises at least one beam deflection unit and/or at least one beam forming unit. The at least two beam guide units are designed for different areas of the kinetic energy of the charged particles such that the at least one beam deflection unit and/or the at least one beam forming unit of each beam guide unit are tailored to the energy of the particles and/or to beam characteristics of the particle beam. The invention further relates to a particle beam treatment method and use of the particle beam treatment system.

The invention relates to a particle beam treatment system comprising a beam generating unit for generating and adjusting the kinetic energy of a beam of charged particles, comprising at least two beam guide units for feeding the beam of charged particles to a treatment location associated with the respective beam guide unit, wherein each beam guide unit comprises at least one beam deflection unit and/or at least one beam forming unit. The invention further relates to a particle beam treatment method and applications of the particle beam treatment system.

Particle beam treatment systems are known from the prior art, e.g. from the patent application US 2005/0139787 A1. Normally, a particle beam is generated in an accelerator and fed to one of the many treatment rooms using beam guide units having magneto-optic equipment. The magneto-optic equipment in the individual treatment rooms is largely identical and designed such that it can feed particle beams having the broadest possible range of particle energy (e.g. from low particle energy of about 70 MeV up to high energies of about 250 Mev; 1 MeV corresponds to approximately 1.6*10⁻¹³ Joules) with the maximum possible transmission efficiency to the treatment location. In the treatment rooms patients can, for instance, precisely exposed for the purpose of tumor therapy.

The transmission efficiency of the particle beam from the accelerator to the respective treatment location essentially depends on the acceptance of the respective magneto-optic equipment on the one hand and on the phase space density of the particle beam on the other. If the particle beam is basically generated by an accelerator having constant energy, e.g. a cyclotron, it could be necessary to reduce the kinetic energy of the particles from the kinetic energy predetermined by the accelerator to a lower kinetic energy by means of a degrader. However, such deceleration methods reduce the phase space density of the particles in the particle beam, which increases the acceptance requirements of the subsequent magneto-optic equipment for the beam line and/or beam forming.

The provision of several treatment rooms next to each other having largely identical beam guide units has the main advantage that treatment plans, which comprise a majority of the patients to be treated, can be drawn up flexibly and in a time-efficient manner. For instance, after the first patient has been treated in the first treatment room, it is possible to prepare this first treatment room for the next treatment, whereas simultaneously, another patient is exposed to the particle beam from the same accelerator in one of the other prepared treatment rooms. The provision of several treatment rooms having independent beam guide units thus has a cost advantage to that extent since the number of patients to be treated can be increased per unit time.

High technical and operating demands are made on the beam guide units for guiding the particle beam from the beam generating unit to the treatment location with a transmission efficiency that is adequate for a treatment. This is particularly applicable to that part of the beam guide unit which is located in a treatment room itself. The magneto-optic equipment required there for the beam line and/or beam forming is mostly installed on a gantry having movable control equipment so as to be able to ensure that a patient kept in the treatment room can be exposed to the particle beam from maximum possible different directions. For this purpose, the gantries are installed in the treatment rooms using the control equipment, normally around a horizontal axis that can be rotated by up to 360°.

A beam line stretching over long beam energy ranges requires magneto-optic equipment having diverse multi-pole (electro) magnets, e.g. dipole magnets, quadrupole magnets and, if applicable, sextupole magnets, which have a suitable spatial extent and a corresponding mass. In addition, the (electro) magnets must be coupled with power supplies that can be adjusted over long ranges so as to be able to generate magnetic field strengths required for the beam line. Basically, the path integral over the magnetic field strength determines the beam line power of the magneto-optic equipment to a large extent.

High magnetic field strengths, which can be generated only using correspondingly designed (electro) magnets, are required for guiding beams having high particle energies, whereas the path integral requirements pertaining to the magnetic field strength are reduced proportionally to the momentum of the particles in case of beams having low particle energies.

Due to the high technical effort required for using such magneto-optic equipment, high demands must also be made on the gantry and its control equipment since they have to provide enough space for the magnets and have sufficient positioning force so as to be able to rotate the heavy magneto-optic equipment quickly and with precision. A gantry having a length of about ten meters and a width of about four meters is an example for feasible dimensions. Hence, the treatment room in which the gantry is installed also requires a high load-carrying capacity so that the gantry can rotate with high precision. The advantage obtained due to the high flexibility when drawing up the treatment plans is reduced again due to technical and operating effort and costs required for using the magneto-optic equipment, which has a largely identical design.

The technology underlying the proton beam transport is also described in detail in the scientific publications mentioned below:
Karl L. Brown, Sam K. Howry, “TRANSPORT, A Computer Program for Designing Charged Particle Beam Transport Systems”, SLAC Report No. 91 (1970) and later updates of the TRANSPORT program by U. Rohrer and others
   U. Rohrer, “PSI Graphic TURTLE Framework based on a CERNSLAC-FERMILAB version by K. L. Brown et al.”, http://people.web.psi.ch/rohrer-u/turtle.htm (2008)
   J. Drees, “Passage of Protons through Thick Degraders”, Cryoelectra Report September 2008

Based on this, the object of this invention is to provide a particle beam treatment system where the technical effort in using the device can be reduced, and thereby the costs associated therewith as well. In addition, the object of this invention is to suggest a preferred method and preferred applications of the particle beam treatment system.

According to the first part of this invention, the object is achieved with a particle beam treatment system in accordance with the preamble of the patent claim 1 such that at least two beam guide units are designed for different ranges of the kinetic energy of the charged particles. They are designed such that at least one beam deflection unit and/or at least one beam forming unit of each beam guide unit is adjusted to the energy of the particles and/or the beam properties of the particle beam.

The described therapy system is based on the notion to use the variability of the beam energy needed for different particle beam therapies for an instrumental and technical simplification of the particle beam treatment system described here

(in other words, the energy of the particles need not be adjusted beyond the maximum necessary energy range for every treatment.) Thus, it is no longer necessary to design the individual beam guide units with their beam deflection units and/or beam forming units in a largely identical manner so that a particle beam having any amount of energy can be controlled optimally by each of the existing beam guide units (i.e. with maximum transmission efficiency). On the contrary, different particle beam energies or beam properties are allocated to the optimized beam guide units and the beam guide units thus have a different design. This results in simplification of the individual beam guide units.

A particle beam emerging from a beam generating unit is not fed to a random beam guide unit from among a majority of equipped beam guide units that are largely identical as regards the design, depending on the energy of its particles and/or depending on its beam properties. Instead, this beam is fed to a beam guide unit whose magneto-optic equipment, particularly its beam deflection unit and/or beam forming unit has been specially optimized for guiding a beam of particles having this energy and/or having these beam properties.

The requirements for the individual beam guide units can thus be reduced in this manner. The multi-pole magnets of such a “specialized” beam guide unit, particularly its beam deflection unit and/or beam forming unit, can be designed such that they have less volume and lower weight.

In addition or as an alternative, even the power supplies to the (electro) magnets of the magneto-optic equipment can be reduced and thus simplified. The gantry control equipment of the magneto-optic equipment simplified in such a manner can thus also be dimensioned such that it is smaller and, if necessary, maneuvered easily, i.e. rotated around the treatment location. A cost reduction for the particle beam treatment system as a whole thus develops from the reduction of the instrumental cost for every single beam guide unit (including beam deflection unit, beam forming unit and, if necessary, power supply and/or gantry).

At least the two beam guide units can have different acceptances in a preferred design of the particle beam treatment system. Acceptance is a parameter that is known from accelerator physics and defines the maximum possible multidimensional phase space, which can be transported to the target location, here the treatment location, by a beam guide system. The phase space represents the spatial extent of the particle beam, its divergence behavior and momentum variance. The phase space occupied by the particles expands to a large extent when slowing down the primary particle beam, e.g. using a degrader. The phase space density falls even more, the more the energy is reduced.

Hence, at least one of the beam guide units can be designed such that it has a high acceptance. This beam guide unit would then be suitable for beams having low particle energies in particular. On the other hand, at least one of the beam guide units can be designed for lower acceptances, in order to reduce the technical effort of the particle beam treatment system as a whole. The beam guide unit having low acceptances would thus be suitable for beams having high particle energies.

In addition, at least one of the beam guide units can be designed such that it has optimum beam transmission. In this advantageous design of the particle beam treatment system, the beam guide unit is designed such that there is least possible loss of particles or beam intensity due to the beam line (in other words, it has maximum possible transmission efficiency). This can also be particularly advantageous when the particle beam has a low phase space density because of a large spatial expansion, especially with a large beam profile.

In addition or as an alternative, at least one of the beam guide units can be designed such that they have optimum beam focusing. In this favorable design of the particle beam treatment system, the beam guide unit is designed such that a strongly focused particle beam having high energy density per unit area can be transmitted to the treatment location.

Such a design of one of the beam guide units can be used to increase the spatial resolution in at least one treatment room in order to reduce the unintentional irradiation of volumes close to the target volume.

The switch-on time of the accelerator can be reduced by designing at least one of the beam guide units such that it has optimum beam transmission and/or beam focusing. In this way, even the accumulated radioactive load of the rooms in which the particle beam treatment system is located can be reduced.

The phase space of the particle beam is determined by ordinary, angular, and momentum space of the particles. Normally, the higher the phase space density, the smaller is the beam profile and the lower is the divergence of the particle beam. A particle beam having high phase space density requires a low beam line effort as compared to a particle beam having low phase space density because the variance of the phase space parameters is higher in the latter case.

Accordingly, the design of the beam guide units is different such that at least one of them is designed for particle beams having high phase space density, by which the technical effort for this beam guide unit is reduced or that at least one of them is designed for particle beams having low phase space density. In the latter case, the instrumental effort cannot be varied arbitrarily, but ensures that even a particle beam having low phase space density can be fed efficiently to the treatment location having adequate transmission efficiency.

The beam generating unit can have an accelerator with adjustable kinetic energy, particularly a synchrotron, in a′ special design of the particle beam treatment system. The energy of the particle beam can be adjusted within a wide range in case of a synchrotron. Since it can be adjusted very accurately, a particle beam generated with a synchrotron generally has a high phase space density.

In an alternative design of the particle beam treatment system, the beam generating unit can have an accelerator with constant kinetic energy, a cyclotron in particular, and an energy correction unit, a degrader in particular. Particle beams only of a particular energy can be generated using a cyclotron that may be super-conductive. Normally, this energy value is set rather high, at 250 MeV for instance. Since such a high particle energy is not required for all the treatment processes, e.g. a treatment of tumors close to the surface requires particles having less kinetic energy, the particle beam can pass through an energy correction unit, e.g. a degrader, after generation in the cyclotron, which adjusts the energy of the particle beam to the desired extent.

The degrader can preferably comprise a material having a low ordinal number Z, where Z is less than 10.

The kinetic energy of the particles can thus be adjusted to the desired extend due to the physical interaction of the particle beam with the degrader material. An advantage of this technology as against synchrotron basically lies in the low operating costs of a cyclotron. A particle beam, which has passed through a degrader, has a low phase space density compared to a particle beam that comes out of a synchrotron, because of the interaction of particles with the degrader material.

The beam of charged particles can be developed as an ion beam, a proton beam in particular. It has been determined that treatment with ions, and particularly with protons as the simplest ions, in tumor therapy on humans is more effective than a treatment with photons for instance. In addition, irradiation with ions, especially protons, is advantageous since these ions show their maximum ionization strength, and thus their maximum destructive power, e.g. for tumor cells, only at the end of their path to the tissues to be irradiated (Bragg Peak). In this way, the effect on the healthy tissue, which is in front of the tissue to be irradiated in the beam path and through which the beam will pass, is reduced.

The beam guide units can have a movable gantry in another advantageous design of the particle beam treatment system.

The gantry can be rotated advantageously, basically around a horizontal axis by up to 360° so as to be able to irradiate the treatment location from maximum possible angles. The gantry also comprises magneto-optic equipment installed on it, at least one beam deflection unit and/or beam forming unit having dipole magnets, quadrupole magnets and, if necessary, additional deflection magnets (e.g. sextupole magnets, . . . ) needed for beam transport and beam forming so as to guide and redirect the particle beam from the beam direction given by the beam generating unit to the treatment location.

The magneto-optic equipment of the individual beam guide units is not identical but is adapted to the energy of the particles and/or the beam properties of the particle beam. This involves reduction in the technical and operating effort of every single beam guide unit, e.g. the weight and/or dimensions of the deflection magnets or the degree of the complexity of the power supplies of the (electro) magnets, by which the instrumental effort of the particle beam treatment system can be reduced as a whole. This also includes an advantageous cost saving.

According to a further intention of this invention, the object is also achieved by a particle beam treatment method, using a particle beam treatment system as described before, in which a beam of charged particles, particularly ions, preferably protons, is generated with a particular amount of energy.

In this, the beam of charged particles is fed to one of at least two beam guide units according to its energy and/or the beam properties, which are designed, basically optimized, for different ranges of the kinetic energy of the protons and in which the proton beam is fed to a treatment location connected to the respective beam guide unit.

The designs of the particle beam treatment system are referred to as regards the advantageous designs of the particle beam treatment method.

Using a particle beam treatment system as described above for irradiating tissues, especially human, is preferred.

Using a particle beam treatment system as described above in tumor therapy, especially on humans, is particularly preferred.

In a medicinal application, it is particularly advantageous to design the individual beam guide units for different energies of the particles and/or beam properties depending on the medicinal requirements and the frequency of the occurring illnesses, which indicate a treatment with particular particle energies and/or beam properties.

Various options are available to design the particle beam treatment system or the particle beam treatment method as per this invention and develop it further. For this, the dependent patent claims on the one hand and the description of an embodiment along with the enclosed drawing on the other are referred to. The drawings show:

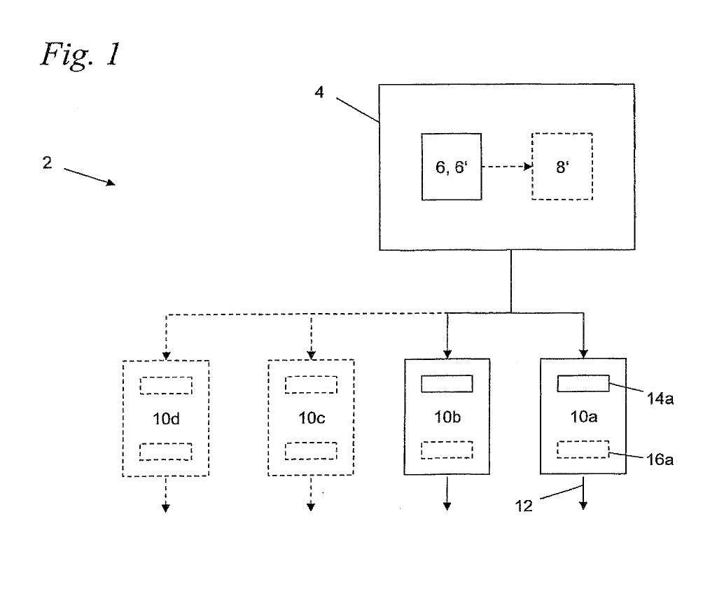

FIG. 1: an embodiment of the particle beam treatment system as per this invention in a schematic view;

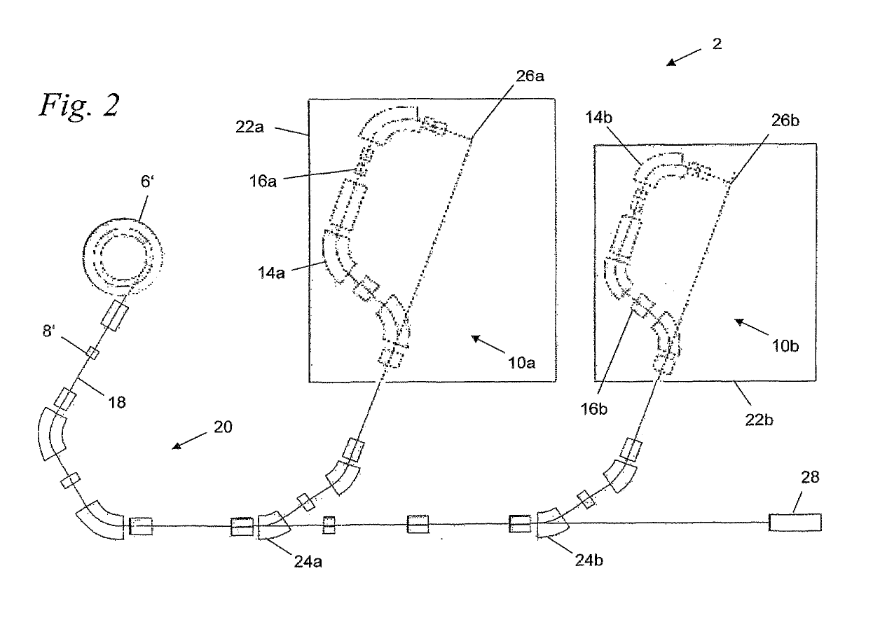

FIG. 2: a schematic overview of an embodiment of the particle beam treatment system as per this invention.

FIG. 1 shows a particle beam treatment system 2 in a schematic view as per this invention. The particle beam treatment system 2 has a beam generating unit 4. The beam generating unit 4 can have an accelerator with adjustable kinetic energy such as a synchrotron 6 for instance.

In an alternative design of the particle beam treatment system, the beam generating unit 4 can also have an accelerator with constant kinetic energy such as cyclotron 6′. The cyclotron 6′ is then preferably designed for providing a particle beam with high energy, e.g. in the range of 200 to 300 MeV, particularly 250 MeV. Since particle beams having such high energy are not required for every treatment, an energy correction unit can be subordinated to the cyclotron 6′.

The energy correction unit can be designed as a degrader 8′ for instance. The particle beam then passes through the degrader 8′ after being emitted by the cyclotron 6′ and is thus slowed down because of the physical interaction between the particles and the degrader material. The extent of slowing down the particle beam in the degrader 8′ can be adjusted.

After the particle beam has been provided with the desired energy by the beam generating unit 4, it is fed to one of the beam guide units 10a, 10b. The beam guide units 10a, 10b are designed for feeding the particle beam to a treatment location (isocenter) allocated to them (arrow 12). The treatment locations are mainly located in different treatment rooms so as to be able to ensure high flexibility when treating a majority of (tumor) patients for instance.

Each of the beam guide units 10a, 10b have at least one beam deflection unit 14a in this example, by which the particle beam can be guided in a particular direction to the respective treatment location. Here only one of them is provided with reference markings to clarify matters. In addition or as an alternative, the beam guide units 10a, 10b can also have beam forming units 16a by which the particle beam can be collimated and/or focused in the beam path for instance, again only one of which is provided with reference markings for clarity.

The serial arrangement of the beam deflection unit 14a and the beam forming unit 16a shown in FIG. 1 is only a schematic representation. It is also possible to interchange the sequence. Likewise, the beam deflection unit 14a and the beam forming unit 16a can have several sub-units (not shown), which can be arranged alternately for instance.

The beam guide units 10a, 10b with their beam deflection units 14a and beam forming units 16a do not have an identical design. On the contrary, the beam guide units 10a, 10b are designed for different kinetic energy ranges of the charged particles of the particle beam. This different design can exist in the form of different acceptances among other things. Due to the different design of the beam guide units 10a, 10b, it is possible to allocate the particle beam generated by the beam generating unit 4 to a specially designed beam guide unit of the beam guide units 10a, 10b depending on the energy of the particles and/or the beam properties. A computer-aided control unit (not shown) of the particle beam treatment system 2 supplies a corresponding allocation algorithm. The energy of the particles or the beam properties can thus be obtained from the adjustments of the beam generating unit 4 and measured using corresponding monitors (not shown) on the particle beam.

This varying allocation can thus be determined by the energy of the particles. If the beam generating unit 4 can generate particles having energy between 70 MeV and 250 MeV, it is possible to divide this energy interval, the first energy interval being e.g. between 100 and 250 MeV and the second between 70 and 200 MeV and each of the beam guide units 10a, 10b can be designed for magneto-optic guiding of particles of the relevant energy interval. In this example, a particle beam, which has an energy of 250 MeV, will be fed to the first beam guide unit 10a and thus to a first treatment location in the first treatment room. While a particle beam, which has an energy of less than 100 MeV, will be fed to the second beam guide unit 10b and thus to a second treatment location in the second treatment room.

Thus, a “specialized” beam guide unit 10a, 10b exists for a particular energy interval. This is particularly advantageous if the entire range of the possible particle energies is not required for a treatment, but only a limited energy range below the maximum particle energy is required. This allows a reduction in the technical effort for the individual beam guide unit 10a, 10b and the beam deflection units 14a and/or beam forming units 16a, in the form of reduced dimensions and less weight of the (electro) magnets or lower degree of complexity of the power supplies (not shown) of the (electro) magnets.

The voltage or current intervals of the energy supplies can be reduced in particular. Thus, a more efficient design of the particle beam treatment system 2 can be obtained.

It is also possible that one of the energy intervals is completely included in another energy interval. This can be advantageous if many treatment cases in a limited energy range are anticipated. For instance, a beam guide unit 10a could be designed for the energy range between 70 and 250 MeV. In this case, the beam guide unit 10a could be used for all the patients and another beam guide unit 10b for a selected number of patients. The advantage of the reduced instrumental effort would then be realized by simplifying the beam guide unit 10b by designing it for a smaller energy interval.

The particle beams can be allocated advantageously to the individual beam guide units 10a, 10b, additionally or alternatively, on the basis of the phase space density, the width of the beam profile or other relevant beam properties in addition to the energy of the particles.

FIG. 2 shows a schematic overview of a particle beam treatment system 2. The particle beam treatment system 2 has an accelerator in the form of a cyclotron 6′. An energy correction unit in the form of a degrader 8′ is located in the beam line for adjusting the energy of the particle beam, provided the maximum energy is not required.

After passing through the degrader, the particle beam passes through a beam transfer unit 20 having several magneto-optic elements, which lead the particle beam into a serial arrangement of several, two in this example, treatment rooms 22a, 22b. Thus, branching units 24a, 24b are located at two points on the beam line 18, by which the particle beam can be guided in the direction of the individual treatment rooms 22a, 22b. A beam capturing unit 28 is located in the beam extension unit behind the branching units 24a, 24b for safety reasons. The decision of when to feed a particle beam to which treatment room 22a, 22b is taken—as already explained—on the basis of the energy of the particles and/or beam properties. However, in case of overlapping parameter intervals, even the treatment plan for the individual treatment rooms 22a, 22b can be used for selecting the beam guide units 10a, 10b.

Beam guide units 10a, 10b having magneto-optic equipment in the form of beam deflection units 14a, 14b (e.g. twice at an angle of +/−70°, once at an angle of 90°) and beam forming units 16a, 16b (e.g. for focusing) are installed in the individual treatment rooms 22a, 22b, which ensure that the particle beam is fed to the respective treatment location 26a, 26b as per the specified conditions, e.g. high degree of focusing. The beam deflection units 14a, 14b and/or the beam forming units 16a, 16b are—as already explained—adjusted advantageously to the energy of the particles and/or beam properties such that a particle beam having a certain configuration can be guided efficiently using one of the beam guide units 10a, 10b.

The beam guide units 10a, 10b have preferably rotatable gantries by which the magneto-optic equipment can be rotated at the treatment location 26a, 26b (up to 360°) to set different irradiation angles.

The particle beam treatment system is not restricted to designs having two beam guide units 10a, 10b. Moreover, it can have more than two, e.g. as indicated in FIG. 1 in dotted lines—four beam guide units 10a, 10b, 10c, 10d. In this way, the allocation of the particle beams can be further differentiated depending on the energy of the particles and/or beam properties. A number of treatment rooms 22a, 22b with treatment locations 26a, 26b installed in them corresponding to the number of the beam guide units 10a, 10b, 10c, 10d is also provided in that case. Resuming the previously mentioned example, one of the beam guide units 10a can be designed for the energy interval between 100 and 250 MeV, both the following beam guide units 10b, 10c between 70 and 200 MeV and the last beam guide unit 10d in this resumed example between 70 and 150 MeV.

It is understood that even in this resumed example, the particle beams can be allocated additionally or alternatively on the basis of the phase space density or the width of the beam profile. The special designs, which were illustrated previously on the basis of the particle energy, allows also the allocation of the particle beams to the individual beam guide units 10a, 10b, 10c, 10d in case of the other beam properties.

1-11. (canceled)

12. A proton beam treatment system comprising
a beam generating unit configured to generate and adjust the kinetic energy of a beam of protons; and
at least two beam guide units configured to feed the proton beam to a treatment location associated with the respective beam guide unit,
wherein the beam guide units each comprise a rotatable isocentric gantry, wherein each beam guide unit comprises at least one of at least one beam deflection unit and at least one beam forming unit,
wherein the beam guide units are of instrumentally different design such that they are designed for different ranges of kinetic energy of the protons, and wherein the at least one of the at least one beam deflection unit and the at least one beam forming unit of each beam guide unit is adapted to at least one of the energy of the protons and the beam properties of the proton beam.

13. The proton beam treatment system according to claim 12, wherein the at least two beam guide units have different acceptances.

14. The proton beam treatment system according to claim 12, wherein at least one of the beam guide units is designed for optimum beam transmission.

15. The proton beam treatment system according to claim 13, wherein at least one of the beam guide units is designed for optimum beam transmission.

16. The proton beam treatment system according to claim 12, wherein at least one of the beam guide units is designed for optimum beam focusing.

17. The proton beam treatment system according to claim 13, wherein at least one of the beam guide units is designed for optimum beam focusing.

18. The proton beam treatment system according to claim 14, wherein at least one of the beam guide units is designed for optimum beam focusing.

19. The proton beam treatment system according to claim 12, wherein the beam generating unit has an accelerator with adjustable kinetic energy.

20. The proton beam treatment system according to claim 19, wherein the accelerator is a synchrotron.

21. The proton beam treatment system according to claim 13, wherein the beam generating unit has an accelerator with adjustable kinetic energy.

22. The proton beam treatment system according to claim 14, wherein the beam generating unit has an accelerator with adjustable kinetic energy.

23. The proton beam treatment system according to claim 16, wherein the beam generating unit has an accelerator with adjustable kinetic energy.

24. The proton beam treatment system according to claim 12, wherein the beam generating unit has an accelerator with constant kinetic energy and an energy correction unit.

25. The proton beam treatment system according to claim 24, wherein the accelerator is a cyclotron.

26. The proton beam treatment system according to claim 24, wherein the energy correction unit is a degrader.

27. The proton beam treatment system according to claim 24, wherein the accelerator is a cyclotron, and wherein the energy correction unit is a degrader.

28. The proton beam treatment system according to claim 13, wherein the beam generating unit has an accelerator with constant kinetic energy and an energy correction unit.

29. The proton beam treatment system according to claim 14, wherein the beam generating unit has an accelerator with constant kinetic energy and an energy correction unit.

30. The proton beam treatment system according to claim 16, wherein the beam generating unit has an accelerator with constant kinetic energy and an energy correction unit.
